(19) United States
(12) Patent Application Publication
(10) Pub. No.: US 2026/0013797 A1
(43) Pub. Date: Jan. 15, 2026

(54) Systems and Methods for Screening and Predicting Sepsis

(71) Applicant: Becton, Dickinson and Company, Franklin Lakes, NJ (US)

(72) Inventors: Anusha Alathur Rangarajan, Irvine, CA (US); Zhongping Jian, Irvine, CA (US); Weiqun Chen, Irvine, CA (US)

(21) Appl. No.: 18/995,056

(22) PCT Filed: Jul. 12, 2023
(86) PCT No.: PCT/US2023/027502

§ 371 Date: Jan. 15, 2025

Related U.S. Application Data
(60) Provisional application No. 63/389,577, filed on Jul. 15, 2022.

(51) Int. Cl. A61B 5/00; A61B 5/021; G16H 50/30

(52) U.S. Cl. CPC A61B 5/7275; A61B 5/021; A61B 5/7267; A61B 5/7278; G16H 50/30

(57) ABSTRACT

Systems and methods for assessment of sepsis using a waveform data and/or other patient information are provided. The waveform data corresponds to a signal, for example, from an arterial blood pressure, or any signal proportional to, or derived from the arterial pressure signal. These systems and methods involve extracting hemodynamic data features from the waveform data and entering the hemodynamic data features into predictive computational models, to yield scores that can be utilized to screen for an early indication of sepsis or can be utilized to predict a probability that an individual is experiencing sepsis.

### CROSS REFERENCE TO RELATED APPLICATIONS

This application claims priority to U.S. Provisional Patent Application No. 63/389,577 entitled “Systems and Methods for Screening and Predicting Sepsis,” filed Jul. 15, 2022, the disclosure of which is herein incorporated by reference.

### TECHNOLOGICAL FIELD

The disclosure is generally directed to systems and methods for screening and predicting sepsis, and more specifically for screening and predicting sepsis with hemodynamic data.

### BACKGROUND

Sepsis is a condition of the body when the body has an overreactive response to a pathogenic infection. Sepsis can lead to tissue damage, organ failure, and death. To prevent severe injury, rapid diagnosis and treatment are required.

Patients are diagnosed with sepsis when they develop a set of signs and symptoms related to the sepsis response. An infection alone is not enough to diagnose sepsis, as most pathogenic infections do not result in sepsis and there is no clear correlation of pathogenic infection to the development of sepsis. One method to diagnose sepsis is assessment of the systemic inflammatory response syndrome (SIRS). A positive diagnosis occurs when at least two of the following criteria are met: (1) fever or hypothermia; (2) elevated heart rate (tachycardia); (3) elevated breathe rate (tachypnea); and (4) low or high counts of white blood cells (leukocytosis or leucopenia) or high ratio of band cells (bandemia).

Sepsis progresses to severe sepsis when signs of organ dysfunction are present. Organ dysfunction can be assessed by the sequential organ failure assessment (SOFA). SOFA assesses lung respiration, blood coagulation, liver function, brain function, cardiovascular function, and kidney function, where higher SOFA scores indicate greater organ dysfunction. High SOFA scores associated with a sepsis diagnosis indicate an immediate need to treat the inflammation and the infection to mitigate organ damage and prevent death.

### SUMMARY

Systems and methods for assessing sepsis can comprise utilization of a sensor to generate a waveform data corresponding to an arterial blood pressure, or a signal proportional to, or derived from, the arterial blood pressure. A set of hemodynamic data features can be extracted from the waveform data. The set of extracted hemodynamic data features and/or other clinical information including but not limited to patient demographics, vital signs, and laboratory test results can be utilized in a computational model to screen for sepsis or to predict a probability that a patient is experiencing sepsis.

In some implementations, a computational method is for screening for sepsis. The method comprises receiving waveform data corresponding to an arterial blood pressure, or a signal proportional to, or derived from, the arterial blood pressure, from a sensor applied to a patient. The method comprises extracting a set of hemodynamic data features from the waveform data. The method comprises entering the set of extracted hemodynamic data features into a predictive computational model to yield a screening score of sepsis. The predictive computational model has been trained to screen for sepsis utilizing the set of extracted hemodynamic data features.

In some implementations, the set of extracted hemodynamic data features comprises heart rate, kurtosis of pressure distribution, and sample entropy of the time when systolic MAP is reached.

In some implementations, the set of extracted hemodynamic data features comprises heart rate, arterial tone factor, sample entropy of decay area, dynamic arterial elastance, and approximate entropy of time of systole.

In some implementations, the predictive computational model utilizes an equation to yield the screening score of sepsis.

In some implementations, the equation is:

$$Screening Score = \frac{1}{1 + {e}^{( - ( hr * 0.08 + kurt * 0. 66 + sampEn * 0.23 - 0.87 ) )}} * 100$$

wherein hr is heart rate, kurt is kurtosis of pressure distribution, and sampEn is sample entropy of the time when systolic MAP is reached.

In some implementations, the equation is:

$$Screening Score = \frac{1}{1 + {e}^{( - ( hr * 0.1 - avgK * 0.004 + decAreaSampEn * 0.04 + dynEa * 0.05 - tSysApEn * 2.21 - 8.26 ) )}} * 100$$

wherein hr is heart rate, avgK is arterial tone factor, decAreaSampEn is sample entropy of decay area, dynEa is dynamic arterial elastance, and tSysApEn is approximate entropy of time of systole.

In some implementations, the screening score of sepsis indicates a risk of developing sepsis. The method further comprises further assessing the patient for sepsis complications.

In some implementations, the screening score of sepsis indicates a risk of developing sepsis. The method further comprises monitoring the patient for sepsis complications for a certain period of time.

In some implementations, a computational model is for predicting a probability of a patient experiencing sepsis. The method comprises receiving waveform data corresponding to an arterial blood pressure, or proportional to, or derived from, the arterial blood pressure, from a sensor applied to a patient. The method comprises extracting a set of hemodynamic data features from the waveform data. The method comprises entering the set of extracted hemodynamic data features into a predictive computational model to yield a probability score of sepsis. The predictive computational model has been trained to predict for sepsis utilizing the set of extracted hemodynamic data features.

In some implementations, the set of extracted hemodynamic data features comprises diastolic pressure, heart rate, entropy of inter-beat interval, time from systolic MAP is reached to the dicrotic notch, and entropy of the standard deviation of decay phase.

In some implementations, the set of extracted hemodynamic data features comprises heart rate, approximate entropy of blood pressure waveform, sample entropy of systolic area, approximate entropy of time of systole, and approximate entropy of time of systolic decay.

In some implementations, the equation is:

$$Probability Score = \frac{1}{1 + {e}^{( - ( - D i a * 0.02 + h * r ⋆ 0.08 - 2.06 * enIBI + 2.8 * timeMAP - 0.59 * e nDecay - 5.13 ) )}} * 100$$

wherein Dia is diastolic pressure, hr is heart rate, enIBI is entropy of inter-beat interval, timeMAP is time from systolic MAP is reached to the dicrotic notch, and enDecay is entropy of the standard deviation of decay phase.

In some implementations, the equation is:

$$Probability Score = \frac{1}{1 + {e}^{( - ( hr * 0.07 + ApEnV * 1.85 - areaSampEn * 0.29 - iSysApEn * 2.29 - tDecApEn * 2.2 - 5.45 ) )}} * 100$$

wherein hr is heart rate, ApEnV is approximate entropy of blood pressure waveform, areaSampEn sample entropy of systolic area, tSysApEn is approximate entropy of time of systole, and tDecApEn is approximate entropy of time of systolic decay.

In some implementations, the probability score of sepsis indicates the patient is septic. The method further comprises further assessing the patient for sepsis complications to confirm the probability score.

In some implementations, the probability score of sepsis indicates the patient is septic. The method further comprises administering a treatment to the patient to treat the sepsis.

In some implementations, the method further comprises sensing, using the sensor, the arterial blood pressure.

In some implementations, the sensor is an intra-arterial catheter and a disposable pressure transducer, a pressurized finger cuff and light sensor, or an applanation tonometer.

In some implementations, the set of extracted hemodynamic features comprises at least one of: heart rate, respiration rate, cardiac output, stroke volume, stroke volume variation, vascular tone, contractility, afterload, systemic vascular resistance, systolic pressure, diastolic pressure, mean arterial pressure (MAP), kurtosis of pressure distribution, left ventricular ejection time, time from when systolic MAP is reached to the dicrotic notch, sample entropy of the time when systolic MAP is reached, entropy of inter-beat interval, entropy of the standard deviation of decay phase,

$$\frac{HR}{fSys} , \frac{Dia}{MAP} , \frac{Sys - DIA}{MAP} , \frac{MAP - Dia}{Sys - Dia} , or \frac{dP}{dt} .$$

In some implementations, the method further comprises entering patient clinical information into the model.

In some implementations, the patient clinical information comprises at least one of: patient demographics, patient vital signs, and patient laboratory results.

In some implementations, the predictive computational model is a regression-based model, a classification-based model or an ensembled model.

In some implementations, a patient monitor system is for screening for sepsis via captured waveform data. The patient monitor system comprises a sensor and a computational processing system in operable connection with the sensor. The computational processing system comprises a processing system and a memory system comprising one or more applications that are configured to direct the processor system to receive waveform data corresponding to an arterial blood pressure, or a signal proportional to, or derived from, the arterial blood pressure, from the sensor applied to a patient; extract a set of hemodynamic data features from the waveform data; and enter the set of extracted hemodynamic data features into a predictive computational model to yield a screening score of sepsis. The predictive computational model has been trained to screen for sepsis utilizing the set of extracted hemodynamic data features.

In some implementations, the set of extracted hemodynamic data features comprises heart rate, kurtosis of pressure distribution, and sample entropy of the time when systolic MAP is reached.

In some implementations, the set of extracted hemodynamic data features comprises heart rate, arterial tone factor, sample entropy of decay area, dynamic arterial elastance, and approximate entropy of time of systole.

In some implementations, the predictive computational model utilizes an equation to yield the screening score of sepsis.

In some implementations, the equation is:

$$Screening Score = \frac{1}{1 + {e}^{( - ( hr * 0.08 + kurt * 0.66 * sampEn * 0.23 - 0.87 ) )}} * 100 ,$$

wherein hr is heart rate, kurt is kurtosis of pressure distribution, and sampEn is sample entropy of the time when systolic MAP is reached.

In some implementations, the equation is:

$$Screening Score = \frac{1}{1 + {e}^{( - ( hr * 0.1 - avgK * 0.004 + decAreaSampEn * 0.04 + dynEa * 0.05 - tSysApEn * 2.21 - 8.26 ) )}} * 100$$

wherein hr is heart rate, avgK is arterial tone factor, decAreaSampEn is sample entropy of decay area, dynEa is dynamic arterial elastance, and tSysApEn is approximate entropy of time of systole.

In some implementations, the one or more applications are further configured to direct the processor system to display the screening score of sepsis on a monitor in operable connection with the computational processing system.

In some implementations, the screening score of sepsis indicates a risk of developing sepsis. The one or more applications are further configured to direct the processor system to, upon determining a screening score of sepsis indicates a risk of developing sepsis, providing an alert indicating the risk.

In some implementations, a patient monitor system is for predicting whether a patient is experiencing sepsis via captured arterial pressure. The patient monitor system comprises a sensor and a computational processing system in operable connection with the sensor. The computational processing system comprises a processor system and a memory system comprising one or more applications that are configured to direct the processor system to receive a waveform data corresponding to an arterial blood pressure, or a signal proportional to, or derived from, the arterial blood pressure, from the sensor applied to a patient; extract a set of hemodynamic data features from the waveform data; and enter the set of extracted hemodynamic data into a predictive computational model to yield a probability score of sepsis. The predictive computational model has been trained to predict for sepsis utilizing the set of extracted hemodynamic data features.

In some implementations, the set of extracted hemodynamic data features comprises diastolic pressure, heart rate, entropy of inter-beat interval, time from systolic MAP is reached to the dicrotic notch, and entropy of the standard deviation of decay phase.

In some implementations, the set of extracted hemodynamic data features comprises heart rate, approximate entropy of blood pressure waveform, sample entropy of systolic area, approximate entropy of time of systole, and approximate entropy of time of systolic decay.

In some implementations, the predictive computational model utilizes an equation to yield the probability score of sepsis.

In some implementations, the equation is:

$$Probability Score = \frac{1}{1 + {e}^{( - ( - Dia * 0.02 + hr * 0.08 - 2.06 * enIBI + 2.8 * timeMAP - 0.59 * enDecay - 5.13 ) )}} * 100$$

wherein Dia is diastolic pressure, hr is heart rate, enIBI is entropy of inter-beat interval, timeMAP is time from systolic MAP is reached to the dicrotic notch, and enDecay is entropy of the standard deviation of decay phase.

In some implementations, the equation is:

$$Probability Score = \frac{1}{1 + {e}^{( - ( hr * 0.07 + ApEnV * 1.85 - areaSampEn * 0.29 - tSysApEn * 2.29 - tDecApEn * 2.2 - 5.45 ) )}} * 100$$

wherein hr is heart rate, ApEnV is approximate entropy of blood pressure waveform, areaSampEn sample entropy of systolic area, tSysApEn is approximate entropy of time of systole, and tDecApEn is approximate entropy of time of systolic decay.

In some implementations, the one or more applications are further configured to direct the processor system to display the probability score of sepsis on a monitor in operable connection with the computational processing system.

In some implementations, the probability score of sepsis indicates a risk of developing sepsis. The one or more applications are further configured to direct the processor system to, upon determining the probability score of sepsis indicates the patient is experiencing sepsis, providing an alert indicating that the patient is experiencing sepsis.

In some implementations, the sensor is an intra-arterial catheter and a disposable pressure transducer, a pressurized finger cuff and light sensor, or an applanation tonometer.

In some implementations, the set of extracted hemodynamic features comprises at least one of: heart rate, respiration rate, cardiac output, stroke volume, stroke volume variation, vascular tone, contractility, afterload, systemic vascular resistance, systolic pressure, diastolic pressure, mean arterial pressure (MAP), kurtosis of pressure distribution, left ventricular ejection time, time from when systolic MAP is reached to the dicrotic notch, sample entropy of the time when systolic MAP is reached, entropy of inter-beat interval, entropy of the standard deviation of decay phase,

$$\frac{HR}{fSys} , \frac{Dia}{MAP} , \frac{Sys - DIA}{MAP} , \frac{MAP - Dia}{Sys - Dia} , or \frac{dP}{dt} .$$

In some implementations, the one or more applications are further configured to direct the processor system to enter patient clinical information into the model.

In some implementations, the patient clinical information comprises at least one of: patient demographics, patient vital signs, and patient laboratory results.

In some implementations, the predictive computational model is a regression-based model, a classification-based model, or an ensembled model.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIG. 1 provides an exemplary method for screening for early signs of sepsis or predicting the probability of sepsis from waveform data.

FIG. 2 provides a conceptual illustration of a computational processing system for screening for sepsis.

FIG. 3 provides a conceptual illustration of a computational processing system for predicting the probability of an individual experiencing sepsis.

### DETAILED DESCRIPTION

The current disclosure details systems and methods to evaluate sepsis utilizing hemodynamic data that is derived from a continuous blood pressure sensor. Hemodynamic data features can be derived from a blood pressure waveform and utilized to screen for and/or predict the probability of sepsis. Accordingly, systems and methods can screen for early identification of sepsis in a patient or can predict the probability of the patient being septic. In some implementations, the hemodynamic data features are utilized in a trained computational model for screening for or predicting the probability of sepsis. In some implementations, the hemodynamic data features are utilized in an equation to compute a score for screening for or predicting the probability of sepsis.

Novel systems and methods provide for screening for and/or predicting probability of sepsis utilizing hemodynamic data. Accordingly, sepsis can be initially screened and/or diagnosed without analysis of SIRS criteria, which may be helpful in situations in which analysis of SIRS criteria is not readily available, for example in an emergency department. In some situations, patients are screened for potential risk of developing sepsis, and when high risk is indicated the patient is monitored and/or further assessed for sepsis. In some situations, a patient is predicted to be septic and subsequent confirmation analysis and/or a treatment for sepsis is performed.

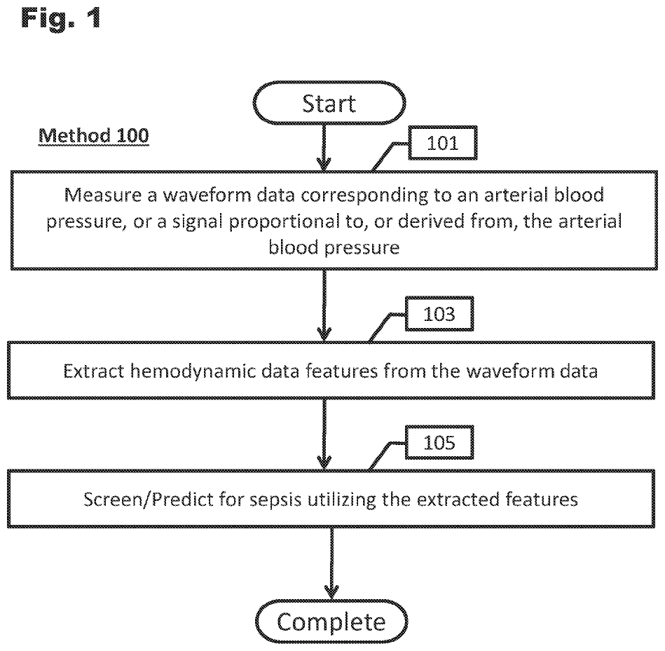

A method for screening for or predicting probability of sepsis is provided in FIG. 1, which can be implemented as a computational process. Method 100 measures (101) a waveform data corresponding to an arterial blood pressure, or a signal proportional to, or derived from, the arterial blood pressure. Any method of measuring continuous arterial blood pressure can be utilized, inclusive of non-invasive and invasive methods. Accordingly, blood pressure can be measured via an intra-arterial catheter (e.g., pressure catheter within an artery) with a disposable pressure transducer, via a pressurized finger cuff and light sensor (e.g., volume clamp method), via applanation tonometry, or any other means that yields an arterial pressure waveform or a signal proportional to, or derived from, the arterial blood pressure.

Method 100 also extracts (103) hemodynamic data features from the waveform data. Various hemodynamic data features are useful for screening for or predicting a probability of sepsis. Generally, any hemodynamic data feature able to provide predictive ability can be utilized. Several hemodynamic data features have been found to provide predictive ability. Hemodynamic data features that can be extracted and utilized to predict or screen for sepsis include (but are not limited to) heart rate, respiration rate, cardiac output, stroke volume, stroke volume variation, vascular tone, contractility, afterload, systemic vascular resistance, systolic pressure, diastolic pressure, mean arterial pressure (MAP), kurtosis of pressure distribution, left ventricular ejection time, time from when systolic MAP is reached to the dicrotic notch, sample entropy of the time when systolic MAP is reached, entropy of inter-beat interval, entropy of the standard deviation of decay phase,

$$\frac{HR}{fSys} , \frac{Dia}{MAP} , \frac{Sys - DIA}{MAP} , \frac{MAP - Dia}{Sys - Dia} , or \frac{dP}{dt} .$$

Method 100 further screens for or predicts (105) the probability of sepsis utilizing the hemodynamic data features extracted from the measured waveform data. In some implementations, the extracted features are entered into a predictive computational model in which the model provides a result that indicates a risk or probability of developing or having sepsis. In some implementations, the extracted features are entered into an equation in which the equation provides a result that indicates a risk or probability of developing or having sepsis. In some implementations, patient clinical information is entered into the model. Clinical information can include (but is not limited to) patient demographics, patient vital signs, and patient laboratory test results.

To screen for or predict a probability of sepsis, a computational model can be trained on hemodynamic data that was collected from a cohort of patients having a known diagnosis of sepsis. The hemodynamic data of each patient can be associated with the patient's sepsis diagnosis to train the model. Various computational models can be utilized, including (but not limited to) regression-based or classification-based models. Regression-based models include (but are not limited to) LASSO regression, ridge regression, k-nearest neighbors, elastic net, least angle regression (LAR), and random forest regression. Classification-based models include (but are not limited to) logistic regression, support vector machines (SVMs), decision trees, random forests, and naïve Bayes. In some implementations, the model is regularized. In some implementations, the model can be ensembled from multiple models from one or more model types listed above.

To screen for or predict a probability of sepsis, an equation can be developed utilizing hemodynamic data that was collected from a cohort of patients having a known diagnosis of sepsis. Weights can be applied to the various hemodynamic data features within the equation to yield a score that provides a diagnostic indication of sepsis.

In one example, a machine learned model has been developed for screening early sepsis identification using features extracted from an arterial blood pressure waveform. The machine learned model developed an equation that utilizes the following features: heart rate (hr), kurtosis of pressure distribution (kurt), and sample entropy of the time when systolic MAP is reached (sampEn). In a particular implementation, the equation is computed as follows:

$$Screening Score = \frac{1}{1 + {e}^{( - ( hr * 0.08 + kurt * 0.66 * sampEn * 0.23 - 0.87 ) )}} * 100 \qquad ( Equation No . 1 )$$

In another example, a machine learned model has been developed for screening early sepsis identification using features extracted from an arterial blood pressure waveform. The machine learned model developed an equation that utilizes the following features: heart rate (hr), arterial tone factor (avgK), sample entropy of decay area (decAreaSampEn), dynamic arterial elastance (dynEa), and approximate entropy of time of systole (tSysApEn). In a particular implementation, the equation is computed as follows:

$$Screening Score = \frac{1}{1 + {e}^{\begin{matrix}( - ( hr * 0.1 - avgK * 0.004 + decAreaSampEn * 0.04 + \\ dynEa * 0.05 - tSysApEn * 2.21 - 8.26 ) )\end{matrix}}} * 100 \qquad ( Equation No . 2 )$$

The screening score indicates an early risk of developing sepsis as provided by a score ranging from 0 to 100. Higher the score means higher the risk of developing sepsis. The features selected, the weights of features, and scaling of score are each provided as an example to yield a screening score of sepsis. Accordingly, the features selected, the weights of features, and scaling of score can be modified, as would be understood in the art.

In some implementations, when a patient's computed screening score indicates a risk of developing sepsis, the patient is further screened for sepsis complications. Further screening can include (but is not limited to) assessment of systemic inflammatory response syndrome (SIRS) criteria, blood lactate concentration, blood culture assessment for bacterial infections, assessment of organ function, and computation of sequential organ failure assessment (SOFA) score. In some implementations, when a patient's computed screening score indicates a risk of developing sepsis, the patient is monitored by a clinician for a certain period of time.

In one example, a machine learned model has been developed for predicting a probability of being septic using features extracted from an arterial blood pressure waveform. The machine learned model developed an equation that utilizes the following features: heart rate (hr), diastolic pressure (Dia), time from systolic MAP is reached to the dicrotic notch (timeMAP), entropy of inter-beat interval (enIBI), entropy of the standard deviation of decay phase (enDecay). In a particular implementation, the equation is computed as follows:

$$Probability Score = \frac{1}{1 + {e}^{\begin{matrix}( - ( - Dia * 0.02 + hr * 0.08 - 2.06 * enIBI + 2.8 * \\ timeMAP - 0.59 * enDecay - 5.13 ) )\end{matrix}}} * 100 \qquad ( Equation No . 3 )$$

In another example, a machine learned model has been developed for predicting a probability of being septic using features extracted from an arterial blood pressure waveform. The machine learned model developed an equation that utilizes the following features: heart rate (hr), approximate entropy of blood pressure waveform (ApEnV), sample entropy of systolic area (areaSampEn), approximate entropy of time of systole (tSysApEn), and approximate entropy of time of systolic decay (tDecApEn). In a particular implementation, the equation is computed as follows:

$$Probability Score = \frac{1}{1 + {e}^{\begin{matrix}( - ( hr * 0.07 + ApEnV * 1.85 - areaSampEn * 0.29 - \\ tSysApEn * 2.29 - tDecApEn * 2.2 - 5.45 ) )\end{matrix}}} * 100 \qquad ( Equation No . 4 )$$

The probability score indicates a probability that an individual is undergoing sepsis as provided by a percentage ranging from 0% to 100%. Higher the percentage means higher the likelihood of experiencing sepsis. The features selected, the weights of features, and scaling of score are each provided as an example to yield a probability score of sepsis. Accordingly, the features selected, the weights of features, and scaling of score can be modified, as would be understood in the art.

In some implementations, when a patient's computed probability score indicates a high probability of being septic, the patient is diagnosed as being septic. In some implementations, when a patient's computed probability score indicates a high probability of being septic, the patient is further screened to confirm the score result. Further screening can include (but is not limited to) assessment of systemic inflammatory response syndrome (SIRS) criteria, assessment of organ function, and computation of sequential organ failure assessment (SOFA) score. When a patient's computed probability score indicates a high probability of being septic, the patient is administered treatments for sepsis. Treatments for sepsis include (but are not limited to) administration of an antibiotic, administration of intravenous fluids, administration of vasopressors, and surgery to remove abscesses, infected tissue or dead tissue.

While specific examples of methods to screen for or predict probability of sepsis are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods to screen for or predict probability of sepsis appropriate to the requirements of a given application can be utilized in various implementations.

### Feature Selection

As explained in the previous section, hemodynamic data and/or other clinical information including but not limited to patient demographics, vital signs, and laboratory test results, are used as features to construct a computational model that is then used to screen for or predict a probability of sepsis. Features used to train the model can be selected by a number of ways. In some situations, features are determined by which data provide strong correlation with a sepsis diagnosis. In some situations, features are determined using a computational model, which can determine which features or feature combinations provide good prediction ability.

Features can be identified and/or selected by several methods. In some instances, features that are relevant based on the clinical significance of sepsis and related ailments are selected. In some instances, features are selected based on high level of correlation with outcome or performance to predict the outcome measure. Accordingly, a strength of relationship between hemodynamic data and a sepsis diagnosis (e.g., SIRS criteria) can be determined. Many statistical methods are known to determine correlation strength (e.g., correlation coefficient), including linear association (Pearson correlation coefficient), Kendall rank correlation coefficient, and Spearman rank correlation coefficient. In some instances, computational models can identify features or feature combinations based on their cost functions. Computational models for selecting features include (but are not limited to) LASSO, elastic net, and ridge regression, which can identify features using weights or coefficients based on their performance. The computational models for identification of useful features can be different (or the same) models than the predictive models used to provide an early screen of sepsis or to predict a probability that a patient is experiencing sepsis. In some instances, a computational approach can search for all possible features and identify which features are most sensitive. Some computational approaches to search for features and identify sensitivity include (but are not limited to) restrictive to recursive feature elimination, and information gain criteria. In some situations, an ensemble approach is utilized that combines multiple models for feature selection and model development. In any approach, an appropriate computational model can be selected that results in a number of features that is manageable. For instance, constructing predictive models from large numbers of features may have overfitting issues. Likewise, too few features can result in less prediction power.

### Computational Processing and Monitoring Systems

A computational processing system to screen for or to predict a probability of sepsis in accordance with the various methods and processes of the disclosure typically utilizes a processing system including one or more of a CPU, GPU and/or neural processing engine. Waveform data corresponding to an arterial blood pressure, or a signal proportional to, or derived from, the arterial blood pressure can be recorded via a sensor. Sensors include (but are not limited to) intra-arterial catheter, a disposable pressure transducer, a pressurized finger cuff and light sensor, and an applanation tonometer. Further, the hemodynamic data features can be extracted from the waveform data to screen for or predict a probability of sepsis.

The computational processing system can be housed within a patient monitor in a direct connection between the monitor and/or components, inclusive of a sensor. Alternatively, the computational processing system can be housed separately from the patient monitor and/or components, receiving the acquired waveform data via a wired or wireless connection (e.g., WiFi, cellular, Bluetooth, etc). The computational processing system can be implemented on any appropriate computing device such as (but not limited to) a patient monitor, a tablet and/or portable computer.

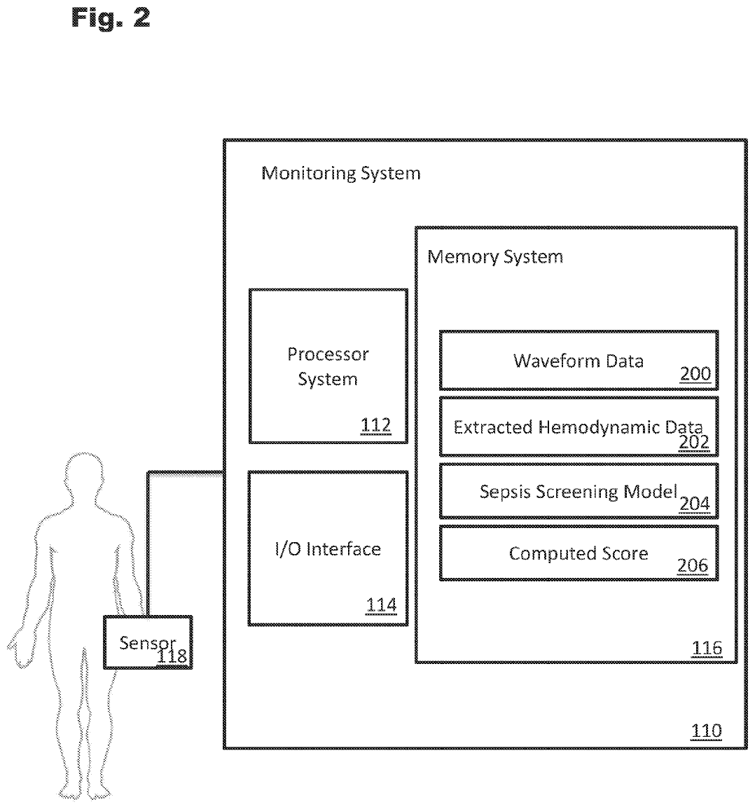

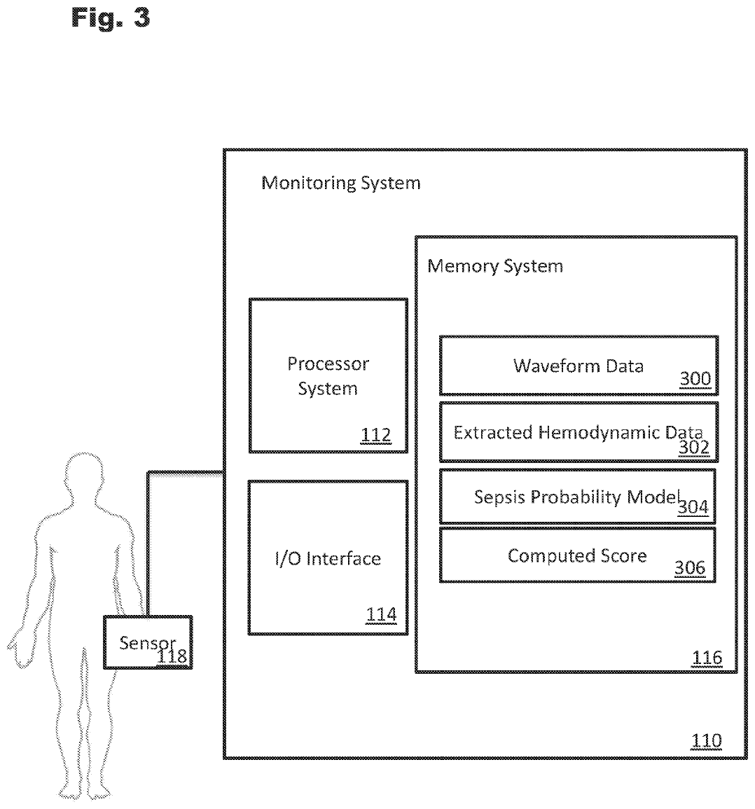

Exemplary computational processing systems that can be utilized to perform the various methods and processes of the disclosure are illustrated in FIGS. 2 and 3. FIG. 2 depicts a computational system to screen for sepsis (e.g., detect the possibility of developing sepsis early) and FIG. 3 depicts a computational system to predict a probability that a patient is experiencing sepsis. Computational processing system 110 includes a processor system 112, an I/O interface 114, a memory system 116, and a sensor 118. As can readily be appreciated, the processor system 112, the I/O interface 114, and the memory system 116 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash).

The sensor 118 can be applied to a patient to sense waveform data of the patient corresponding to an arterial blood pressure, or a signal proportional to, or derived from, the arterial blood pressure. The sensor 118 is operatively in connection with the monitoring system 110 and the I/O interface 114, which can provide a visual representation of the arterial pressure waveform captured from the sensor. The sensor 118 can be a noninvasive or an invasive pressure sensor. Accordingly, the sensor 118 can be an intra-arterial catheter (e.g., pressure catheter within an artery) with a disposable pressure transducer, a pressurized finger cuff and light sensor (e.g., volume clamp method), an applanation tonometer, or any other pressure sensor that yields an arterial pressure waveform.

In the illustrated example, the memory system 116 is capable of storing various data and models. It is to be understood that the listed data and models are a representative sample of what can be stored in memory and that various memory systems may store some or all of the various data and models listed. Further, any combination of data and models can be stored, and in some implementations, various data, applications, and/or models are stored temporarily.

In some implementations, the memory system 116 can store the waveform data 200, which can be obtained from the sensor 118. An application can extract hemodynamic data 202 from the waveform data 200, which can also be stored in memory system 116. The extracted hemodynamic data 202 can be utilized in a sepsis screening model 204, which can be stored in the memory system 116. A processor system 112 is configured to execute the sepsis screening model 204 to generate a computed score 206 indicative of early screening of sepsis of a patient. Further, the waveform data 200 and/or the computed score 206 can be displayed on a monitor or other screen via the I/O interface 114.

In some implementations, the memory system 116 can store the waveform data 300, which can be obtained from the sensor 118. An application can extract hemodynamic data 302 from the waveform data 300, which can also be stored in memory system 116. The extracted hemodynamic data 302 can be utilized in a sepsis probability model 304, which can be stored in memory system 116. A processor system 112 is configured to execute sepsis probability model 304 to generate a computed score 306 indicative of a probability of the patient having sepsis. Further, the waveform data 300 and/or the computed score 306 can be displayed on a monitor or other screen via the I/O interface 114.

Based on the computed score, the monitoring system 110 can provide an alert to clinicians of a screening result and/or septic probability result. Especially in cases in which an individual is predicted to be septic, an alert can enable timely and effective intervention to prevent organ failure or other severe complications associated with sepsis.

While specific computational processing systems are described above with reference to FIGS. 2 and 3, it should be readily appreciated that computational processes and/or other processes utilized in the provision of sepsis screening or prediction can be implemented on any of a variety of processing devices including combinations of processing devices. Accordingly, computational devices should be understood as not limited to specific monitoring systems, computational processing systems, and/or specific applications and models. Computational devices can be implemented using any of the combinations of systems described herein and/or modified versions of the systems described herein to perform the processes, combinations of processes, and/or modified versions of the processes described herein.

1. A computational method for screening for sepsis, comprising:
receiving waveform data corresponding to an arterial blood pressure, or a signal proportional to, or derived from, the arterial blood pressure, from a sensor applied to a patient;
extracting a set of hemodynamic data features from the waveform data; and
entering the set of extracted hemodynamic data features into a predictive computational model to yield a screening score of sepsis, wherein the predictive computational model has been trained to screen for sepsis utilizing the set of extracted hemodynamic data features.

2. The computational method of claim 1 further comprising:
sensing, using the sensor, the arterial blood pressure.

3. The computational method of claim 1, wherein the sensor is an intra-arterial catheter and a disposable pressure transducer, a pressurized finger cuff and light sensor, or an applanation tonometer.

4. The computational method of claim 1, wherein the set of extracted hemodynamic features comprises at least one of: heart rate, respiration rate, cardiac output, stroke volume, stroke volume variation, vascular tone, contractility, afterload, systemic vascular resistance, systolic pressure, diastolic pressure, mean arterial pressure (MAP), kurtosis of pressure distribution, left ventricular ejection time, time from when systolic MAP is reached to the dicrotic notch, sample entropy of the time when systolic MAP is reached, entropy of inter-beat interval, entropy of the standard deviation of decay phase
$$\frac{HR}{fSys} , \frac{Dia}{MAP} , \frac{Sys - DIA}{MAP} , \frac{MAP - Dia}{Sys - Dia} , or \frac{dP}{dt} .$$

5. The computational method of claim 1, further comprising entering patient clinical information into the model.

6. The computational method of claim 5, wherein the patient clinical information comprises at least one of: patient demographics, patient vital signs, and patient laboratory results.

7. The computational method of claim 1, wherein the set of extracted features comprises heart rate, kurtosis of pressure distribution, and sample entropy of the time when systolic MAP is reached.

8. The computational method of claim 1, wherein the set of extracted features comprises heart rate, arterial tone factor, sample entropy of decay area, dynamic arterial elastance, and approximate entropy of time of systole.

9. The computational method of claim 1, wherein the predictive computational model utilizes an equation to yield the screening score of sepsis.

10. The computational method of claim 9, wherein the equation is:
$$Screening Score = \frac{1}{1 + {e}^{( - ( hr * 0.08 + kurt * 0.66 * sampEn * 0.23 - 0.87 ) )}} * 100$$
wherein hr is heart rate, kurt is kurtosis of pressure distribution, and sampEn is sample entropy of the time when systolic MAP is reached.

11. The computational method of claim 9, wherein the equation is:
$$Screening Score = \frac{1}{1 + {e}^{( - ( hr * 0.1 - avgK * 0.004 + decAreaSampEn * 0.04 + dynEa * 0.05 - tSysApEn * 2.21 - 8.26 ) )}} * 100$$
wherein hr is heart rate, avgK is arterial tone factor, decAreaSampEn is sample entropy of decay area, dynEa is dynamic arterial elastance, and tSysApEn is approximate entropy of time of systole.

12. The computational method of claim 1, wherein the predictive computational model is a regression-based model, a classification-based model or an ensembled model.

13. The computational method of claim 1, wherein the screening score of sepsis indicates a risk of developing sepsis; the method further comprising:
further assessing the patient for sepsis complications.

14. The computational method of claim 1, wherein the screening score of sepsis indicates a risk of developing sepsis; the method further comprising:
monitoring the patient for sepsis complications for a certain period of time.

15. A computational method for predicting a probability of a patient experiencing sepsis, comprising:
receiving waveform data corresponding to an arterial blood pressure, or proportional to, or derived from, the arterial blood pressure, from a sensor applied to a patient;
extracting a set of hemodynamic data features from the waveform data; and
entering the set of extracted hemodynamic data features into a predictive computational model to yield a probability score of sepsis, wherein the predictive computational model has been trained to predict for sepsis utilizing the set of extracted hemodynamic data features.

16. The computational method of claim 15 further comprising:
sensing, using the sensor, the arterial blood pressure.

17. The computational method of claim 15, wherein the sensor is an intra-arterial catheter and a disposable pressure transducer, a pressurized finger cuff and light sensor, or an applanation tonometer.

18. The computational method of claim 15, wherein the set of extracted hemodynamic features comprises at least one of: heart rate, respiration rate, cardiac output, stroke volume, stroke volume variation, vascular tone, contractility, afterload, systemic vascular resistance, systolic pressure, diastolic pressure, mean arterial pressure (MAP), kurtosis of pressure distribution, left ventricular ejection time, time from when systolic MAP is reached to the dicrotic notch, sample entropy of the time when systolic MAP is reached, entropy of inter-beat interval, entropy of the standard deviation of decay phase
$$\frac{HR}{fSys} , \frac{Dia}{MAP} , \frac{Sys - DIA}{MAP} , \frac{MAP - Dia}{Sys - Dia} , or \frac{dP}{dt} .$$

19. The computational method of claim 15, further comprising entering patient clinical information into the model.

20. The computational method of claim 19, wherein the patient clinical information comprises at least one of: patient demographics, patient vital signs, and patient laboratory results.

21-54. (canceled)
